(19) United States
(12) Patent Application Publication
(10) Pub. No.: US 2026/0325692 A1
(43) Pub. Date: Oct. 22, 2026

(54) DRAIN DISINFECTION SYSTEMS AND METHODS

(71) Applicant: Kohler Co., Kohler, WI (US)

(72) Inventor: Yachna GARG, Gurgaon (IN)

(21) Appl. No.: 19/653,242

(22) Filed: Apr. 21, 2026

Related U.S. Application Data
(60) Provisional application No. 63/792,087, filed on Apr. 21, 2025.

(51) Int. Cl. A61L 2/104; A61L 2/202; A61L 2/24

(52) U.S. Cl. CPC A61L 2/104; A61L 2/202; A61L 2/24; A61L 2202/11; A61L 2202/121; A61L 2202/14; A61L 2202/15

(57) ABSTRACT

Drain systems having sanitization or disinfection functionality to eliminate potentially infectious microorganisms. Examples of the present disclosure provide drain disinfection systems including a disinfection module configured to sanitize a drain by eliminating microorganisms inside the drain or a drain passageway. The disinfection module can be an ultraviolet light-emitting component, an electric cell configured to output low-intensity electric pulses, an ozone generator configured to release ozone into fluid flowing through the drain, or an electrolyzation cell configured to electrolyze fluid flowing through the drain, among other disinfection technologies. The disinfection module can be powered by a power unit and controlled by a controller. The disinfection module can be positioned proximate a p-trap section of a drain passageway to eliminate microorganisms growing on the p-trap section and present in fluids flowing through the passageway.

### RELATED APPLICATIONS

This application claims the benefit of U.S. Provisional Application No. 63/792,087 filed Apr. 21, 2025, which is hereby fully incorporated herein by reference.

### TECHNICAL FIELD

The present disclosure generally relates to drain disinfection. More specifically, the present disclosure relates to drain systems having sanitization or disinfection functionality to eliminate potentially infectious microorganisms growing within a drain passageway.

### BACKGROUND

Drains direct wastewater away from sinks, showers, and other fixtures in residential, commercial, and industrial plumbing systems. Over time, drains can become breeding grounds for microorganisms, including bacteria, fungi, and other pathogens. These microorganisms often form biofilms on the surfaces of pipes, creating persistent colonies that are resistant to conventional cleaning methods. Water flowing through these contaminated drains can generate aerosols that carry harmful microorganisms into the surrounding environment. This can pose significant health and safety risks to occupants. Such aerosolization is particularly concerning in kitchens, bathrooms, and healthcare facilities, where maintaining a high standard of hygiene is essential.

Traditional approaches to drain cleaning, such as manual scrubbing, chemical disinfectants, or periodic flushing, are labor-intensive, environmentally harmful, or are only temporarily effective. Many of these methods also fail to address the root cause of contamination: the persistent biofilms that adhere to drain surfaces. Biofilms not only shield microorganisms from disinfection efforts but can also facilitate the release of pathogens into the air and water. Continuous and effective disinfection of drains without compromising user safety or convenience would be advantageous. Such solutions must be capable of addressing both surface and waterborne contaminants while minimizing human exposure to harmful substances or processes.

### SUMMARY

Examples of the present disclosure provide drain disinfection systems including a disinfection module configured to sanitize a drain by eliminating microorganisms inside the drain, a power unit configured to supply power to the disinfection module, and a controller configured to control operation of the disinfection module. The disinfection module can be positioned proximate a p-trap section of a drain passageway to eliminate microorganisms growing on or in the p-trap section and present in fluids flowing through the drain passageway. The entire drain passageway, including the p-trap section, is effectively sanitized as fluid flows through the passageway.

Examples of the present disclosure provide drain disinfection systems including a disinfection module including at least one ultraviolet light-emitting component configured to emit ultraviolet light to sanitize a drain by eliminating microorganisms inside the drain, and a power unit configured to supply power to the disinfection module.

Examples of the present disclosure provide drain disinfection systems including an electric cell configured to output low-intensity electric pulses to eliminate microorganisms from a drain, and a power unit configured to supply power to the electric cell.

Examples of the present disclosure provide drain disinfection systems including an ozone generator configured to release ozone into fluid flowing through a passageway of a drain to eliminate microorganisms in the passageway using the ozonated fluid, and a power unit configured to supply power to the ozone generator.

Examples of the present disclosure provide drain disinfection systems including an electrolyzation cell configured to electrolyze fluid flowing through a passageway of a drain to eliminate microorganisms in the passageway using the electrolyzed fluid, and a power unit configured to supply power to the electrolyzation cell.

The above summary is not intended to describe each illustrated example or every implementation of the subject matter hereof. The figures and the detailed description that follow more particularly exemplify various examples.

### BRIEF DESCRIPTION OF THE DRAWING

The present disclosure may be more completely understood in consideration of the following detailed description of examples of the disclosure in connection with the accompanying drawing, in which:

FIGS. 1A and 1B are perspective views of a drain disinfection system used with a basin and a faucet, according to examples of the present disclosure;

FIG. 2 is a side view of the drain disinfection system of FIGS. 1A and 1B;

FIG. 3 is a side view of a disinfection module used with the drain disinfection system of FIGS. 1A-2;

FIG. 4A is a perspective view detailing the power unit used with the drain disinfection system of FIGS. 1A-2;

FIG. 4B is a perspective view detailing the disinfection module of FIG. 3 used with the drain disinfection system of FIGS. 1A-2;

FIG. 5A is a perspective view of the drain disinfection system of FIGS. 1A-2;

FIG. 5B is a perspective view detailing the disinfection module of FIGS. 3 and 4B used with the drain disinfection system of FIGS. 1A-2 and 5A;

FIG. 6A is a perspective view of a drain disinfection system used with a basin and a faucet, according to examples of the present disclosure;

FIG. 6B is a side view of the drain disinfection system of FIG. 6A;

FIG. 7 is a perspective view of a drain disinfection system used with a basin and a faucet, according to examples of the present disclosure; and

FIG. 8 is a perspective view of a drain disinfection system used with a basin and a faucet, according to examples of the present disclosure.

While various examples are amenable to various modifications and alternative forms, specifics thereof have been shown by way of example in the drawings and will be described in detail. It should be understood, however, that the intention is not to limit the claimed inventions to the particular examples described. On the contrary, the intention is to cover all modifications, equivalents, and alternatives falling within the spirit and scope of the subject matter as defined by the claims.

### DETAILED DESCRIPTION OF THE DRAWING

The following detailed description should be read with reference to the figures in which similar or identical elements in different figures are numbered the same. The figures, which are not necessarily to scale, depict illustrative examples and are not intended to limit the scope of the disclosure. It should also be understood that the terminology used herein is for the purpose of description only and should not be regarded as limiting. Common definitions are generally intended for the terminology used herein unless a specific definition is provided. In the case of a term having a common definition and a specific definition provided herein, the term should be construed according to its specific definition.

Examples of the present disclosure include drain disinfection systems and drain disinfecting or sanitizing techniques used with sink systems having faucets and basins, among other components. Example sink systems compatible with the present disclosure include those found in residential settings, commercial settings, and industrial settings (e.g., kitchens, baths, showers, lavatories), or any other setting where dirtied fluid and other waste flows downward through a drain. The drain disinfection systems and techniques provided herein are designed to eliminate microorganisms within sink drains, thereby preventing biofilm formation and the release of aerosols into the surrounding environment. These systems and techniques can include different disinfection techniques, such as ultraviolet light, low-intensity electric pulses, ozone generation, and electrolyzed fluid. Each disinfection technique is tailored to sanitize drain surfaces and fluids effectively while eliminating or reducing aerosolization outside the drain.

For example, drain disinfection systems can use ultraviolet light-emitting diodes or other lighting components to disinfect the “p-trap” section of the drain, or can employ electric pulses to dislodge microorganisms therein. Additional systems can use ozonated or electrolyzed water to neutralize pathogens within the drain. Systems of the present disclosure are designed to maximize user convenience and safety by incorporating features such as programmable controllers for tailored disinfection schedules, sensors to detect fluid flow or contamination levels, and display units to monitor system performance. The systems also prioritize compact and waterproof designs to integrate seamlessly with sink components while protecting the electrical or mechanical parts they include. Sediment filters for pre-cleaning fluids and indicators to signal operational status are also provided with examples of the present disclosure. Collectively, these systems provide versatile and effective solutions for maintaining hygiene and reducing health risks in sink systems by eliminating difficult-to-reach microorganisms growing inside drains.

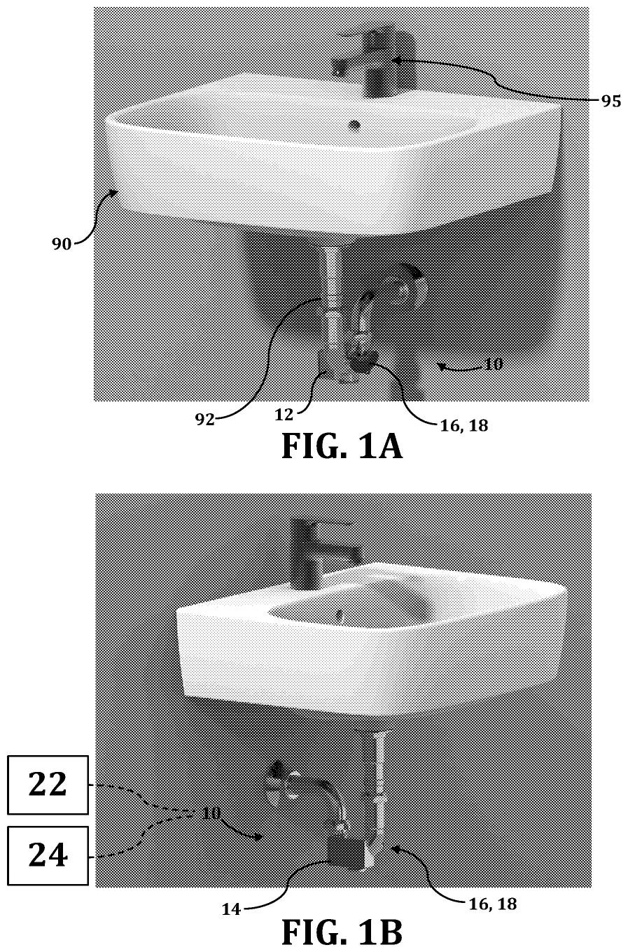

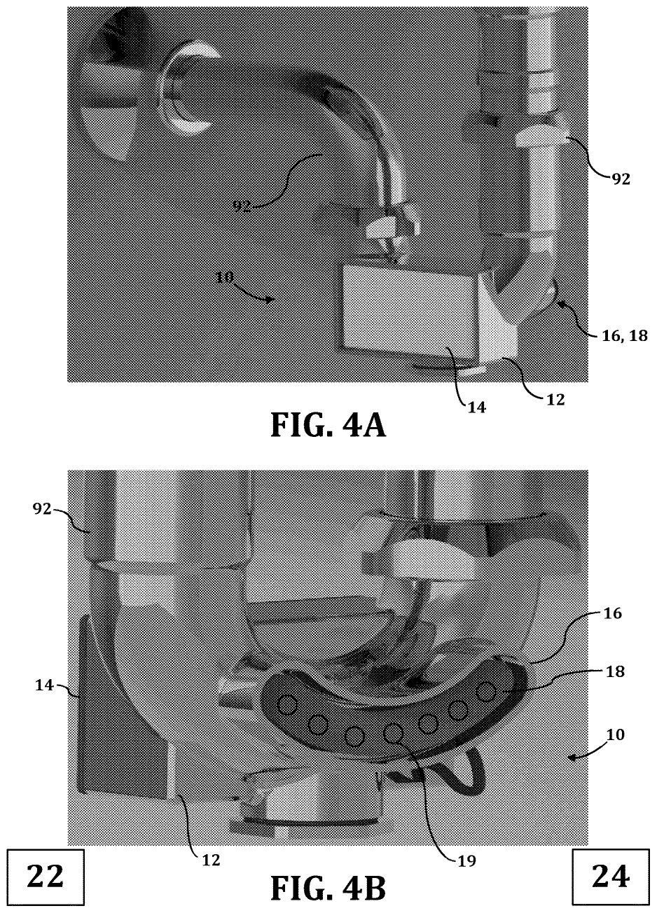

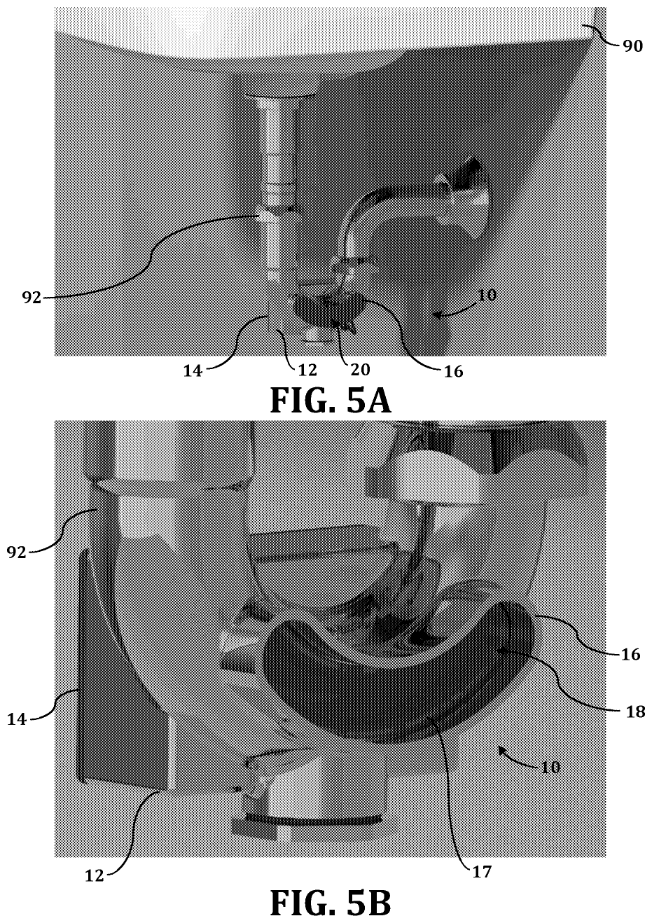

FIGS. 1A-5B depict a drain disinfection system 10 used with a basin 90 and a faucet 95, according to examples of the present disclosure. Drain disinfection system 10 can eliminate potentially infectious microorganisms located in a drain or drain passageway which, if left untreated, can grow over time and form a biofilm within the drain. When water impacts the biofilm, aerosols containing the infectious microorganisms can be released outside the drain and into the environment surrounding the basin and faucet of a sink system. The aerosolized, potentially infectious microorganism dispersion can cause infections or other ailments in individuals that use the sink system, for example to consume water from the faucet or to clean dishware inside the basin. The disinfection and sanitization functionality included with drain disinfection system 10 (and other examples of the present disclosure) remedies these issues.

Drain disinfection system 10 can include a power unit 14 received within a first housing 12 couplable to or integrally formed with drain piping or tubing 92 of a sink system (e.g., sink system including system 10, basin 90, and faucet 95, among other components). Drain disinfection system 10 can further include a disinfection module 18 received within a second housing 16 couplable to or integrally formed with piping or tubing 92 of the sink system. In some examples, first housing 12 may be positioned on an opposite side of piping or tubing 92 relative to second housing 16. In other examples, first housing 12 may be positioned on the same side or in the same region of piping or tubing 92 as second housing 16. In other examples, first and second housings 12, 16 may form a unitary housing for receiving power unit 14 and disinfection module 18 concurrently. First and second housings 12, 16 are generally waterproof. This protects the power unit 14 and the disinfection module 18 from fluids flowing through the drain. While drain disinfection system 10 is depicted in the Figures as being coupled to or formed with the p-trap section of drain piping, it will be understood that drain disinfection system 10 can also be coupled to or formed with the tailpiece or wall tube sections of drain piping.

Power unit 14 can be configured to supply electrical or mechanical power to the disinfection module 18 (and other components of system 10) for operation. In some examples, power unit 14 can be a battery or battery pack that is rechargeable (e.g., wired or wireless charging) or replaceable with a new battery or battery pack. In other examples, power unit 14 can be an electrical motor or a mechanical motor, hydraulically or pneumatically powered, powered by a renewable energy source, or capable of outputting power received from an external power source used with sink systems. Power unit 14 can be configured to continuously output power to disinfection module 18 and other components of system 10 until turned off. In some examples, power unit 14 can include a device to store energy for future use with system 10.

Power unit 14 can be coupled to drain piping or tubing 92 of the sink system via first housing 12. When first housing 12 is integrated within piping or tubing 92, power unit 14 can be received therein in the same manner but with sufficient spacing or coverage away from fluids transiting the piping or tubing 92. This prevents power unit 14 from being wetted or otherwise impacted by fluids during normal operation of the sink system. In some examples, first housing 12 can have a rectangular or substantially rectangular geometry with an opening for receiving the power unit 14. In other examples, first housing 12 can have a different regular or irregular polygonal geometry suited to receive power unit 14 (e.g., spherical, cylindrical, irregular polygonal, and other similar geometries).

First housing 12 can be integrated with piping or tubing 92 such that the components form a single unitary component for receiving power unit 14 and for transmitting fluids through sink system (but with these functions separated or spaced apart). The opening through which power unit 14 is received within first housing 12 can be covered with a panel or shield (not explicitly depicted) to protect power unit 14 from the outside environment. In some examples, this panel or shield can be at least partially transparent to view power unit 14 from the outside. The panel or shield can be removably coupled to first housing 12 so that power unit 14 can be accessed as needed (e.g., for charging or replacement with a new power unit 14).

Disinfection module 18 can be configured to sanitize or disinfect drains and drain passageways by eliminating microorganisms located therein. Potentially infectious microorganisms that can be eliminated with disinfection module 18 include Escherichia coli (E. coli), Salmonella, Listeria monocytogenes, Campylobacter, Staphylococcus aureus, Pseudomonas aeruginosa, Klebsiella spp., Clostridium perfringens, Enterococcus spp., and Shigella, among others. In some examples, disinfection module 18 can also be configured to filter contaminants from fluids transiting the drain passageway, for example if such fluids are intended to be reused (e.g., reused in human-facing applications).

In some examples, disinfection module 18 can be received within a second housing 16 couplable to drain piping or tubing 92 of the sink system. In some examples, second housing 16 can have a slotted geometry with an opening for receiving the disinfection module 18. In other examples, second housing 16 can have a different regular or irregular polygonal geometry suited to receive disinfection module 18 (e.g., a geometry to match the configuration or design of module 18). Second housing 16 can be integrated with piping or tubing 92 such that the components form a single unitary component for receiving disinfection module 18 and for transmitting fluids through sink system (but with module 18 positioned to enable disinfection or sanitization of the drain or drain passageway). The opening through which disinfection module 18 is received within second housing 12 can be covered with a panel or shield 17 to protect module 18 from the water flowing through the tubing 92. In some examples, panel or shield 17 can be at least partially transparent to view so that the light can pass from disinfection module 18 into the drain piping. A second panel or cover 20 can be removably coupled to second housing 12 to protect module 18 from the outside environment. Panel or cover 20 is removable so that disinfection module 18 can be accessed (e.g., for repair or replacement).

In some examples, disinfection module 18 can be designed as an elongated strip including a printed circuit board (PCB) with various electrical connections (e.g., connections to power unit 14). The elongated PCB strip can be sized to fit proximate to or within a “p-trap” section of drain piping or tubing 92 (e.g., can include length, width, height, diameter, or other measurements compatible with the p-trap or other piping or tubing 92 section where disinfection module is positioned). This is depicted particularly in FIGS. 1A, 3, 4B, and 5B. In such examples, disinfection module 18 can include a plurality of ultraviolet light-emitting diodes (LEDs) or other lighting components 19 arranged along the elongated PCB strip. The ultraviolet LEDs or other lighting components 19 can be arranged in any suitable or configuration, and may be equidistantly or non-equidistantly arranged in examples. In some examples, the plurality of LEDs or other lighting components 19 can be arranged as one or more arrays of LEDs or other lighting components (e.g., several LED groupings in multiple spots along the elongated PCB strip).

The plurality of LEDs or other lighting components 19 can be configured to emit ultraviolet light to eliminate microorganisms growing in the drain or drain passageway of a sink system. Ultraviolet light is known to have a strong disinfection rate and can have a residual effect on the disinfected or sanitized areas as well. Because the disinfection module 18 is generally located proximate to or within the “p-trap” section of drain piping or tubing 92, which is located away from the drain hole, human exposure to ultraviolet light is reduced or prevented entirely. Disinfection module 18 can disinfect or sanitize both the drain surface and fluids in the drain using the plurality of LEDs or other lighting components 19. This prevents the aerosolization of potentially infectious microorganisms into the environment outside the drain.

In some examples, the elongated strip can comprise an ultraviolet C-LED strip. In some examples, disinfection module 18 can be programmed to be turned on or off at particular times of the day or when certain circumstances exist (e.g., whenever fluid is flowing through the drain passageway). Programming can be achieved using a controller 22 coupled (e.g., operably, electrically, communicatively) to the disinfection module 18. Controller 22 can be located external to the drain or sink system, and can be operated by an external power source (e.g., external battery) or by power unit 14.

In some examples, disinfection module 18 can include one or more sensors 21 (e.g., proximity or motion sensors, photo interrupters) configured to sense when fluid flows through the drain. Disinfection module 18 can be turned on via controller 22 to commence disinfecting or sanitizing operations when the sensor(s) 21 sense fluid flowing through the drain. In some examples, the sensor(s) 21 can include a contamination or growth sensor configured to sense when microorganism contamination or growth has exceeded a certain level or amount (e.g., a level or amount preprogrammed with disinfection module 18). When this happens, controller 22 can communicate an instruction to turn on disinfection module 18 to commence disinfecting or sanitizing operations. In some examples, the magnitude of disinfection or sanitization using the plurality of LEDs or other lighting components 19 can be adjusted by controller 22 to account for variations in microorganism type, growth, location, and other related characteristics. The present disclosure contemplates that the ultraviolet spectrum will not affect performance of the sensor(s) 21 which can be configured to operate in the visual spectrum as well.

In some examples, the plurality of LEDs or other lighting components 19 can operate in a wavelength range of approximately 180 to 222 nanometers in some examples, or approximately 260 to 280 nanometers in other examples. Other wavelength ranges are also contemplated for use with examples of the present disclosure. In some examples, disinfection module 18 can be programmed to output a minimum or maximum ultraviolet dose using the plurality of LEDs or other lighting components 19. The minimum or maximum dose may be per LED or lighting component, or for all LEDs and lighting components in combination. For example, a minimum ultraviolet dose can be 30 mJ/cm² (units of light energy per unit area) or a value less than or greater than 30. A maximum ultraviolet dose can be substantially above 30 mJ/cm² in some examples. In some examples, disinfection module 18 can be designed to withstand maximum operating temperatures of about 39±3 °C (about 102±5 °F). In some examples, the plurality of LEDs or other lighting components 19 can operate with a power of about 20 to 70 milliwatts (mW).

In some examples, the plurality of LEDs or other lighting components 19 are arranged an alternating pattern of wavelengths to vary the intensity of ultraviolet light emitted from the disinfection module 18. For example, a first lighting component 19 can operate in a wavelength range of approximately 180 to 222 nanometers (nm), while an adjacent lighting component 19 can operate in a wavelength range of approximately 260 to 280 nanometers. This every-other alternating pattern can be provided along module 18. The lighting components at different wavelengths can be operated simultaneously, or in alternating patterns, e.g., LEDs at a first wavelength are on when LEDs at a second wavelength are off, and vice versa, in a pulsing pattern. Any of variety of other patterns can be contemplated, e.g., two lighting components at a first wavelength, a third at a second wavelength, then two lighting components at the first wavelength, etc.

In other examples, the plurality of LEDs or other lighting components 19 can activate in an alternating pattern to vary the intensity of ultraviolet light emitted from the disinfection module 18. For example, if the plurality of LEDs 19 includes eight LEDs, then the alternating pattern could have four of the LEDs activate simultaneously while the remaining four LEDs stay inactivated. The remaining four LEDs could then be activated while the previous four LEDs become inactivated. This provides an alternating arrangement of ultraviolet emission from the eight LEDs. Alternating patterns of ultraviolet light can provide a higher rate of disinfection or sanitization in certain scenarios. In other examples, the plurality of LEDs or other lighting components 19 can include less than or more than eight components that can be activated with a variety of alternating arrangements (e.g., ten LEDs in groups of four and six, the four LEDs activate while the six LEDs are inactivated, and vice versa). In some examples, the LED or other lighting component activation arrangement can be varied based on activation time or emission wavelength to provide customization to drain disinfection or sanitization operations.

Drain disinfection system 10 can further include a display unit 24 that can indicate the functioning of power unit 14, disinfection module 18, and controller 22. For example, display unit 24 can output various disinfection characteristics such as whether the disinfection module 18 is currently operating, when it last operated, statistics on microorganism elimination, system operating mode, ultraviolet LED or other lighting component arrangement or pattern, operational errors, and other similar characteristics. Display unit 24 can be positioned external to the sink system, for example on a countertop near faucet 95, or can be positioned below a sink deck proximate drain piping or tubing 92.

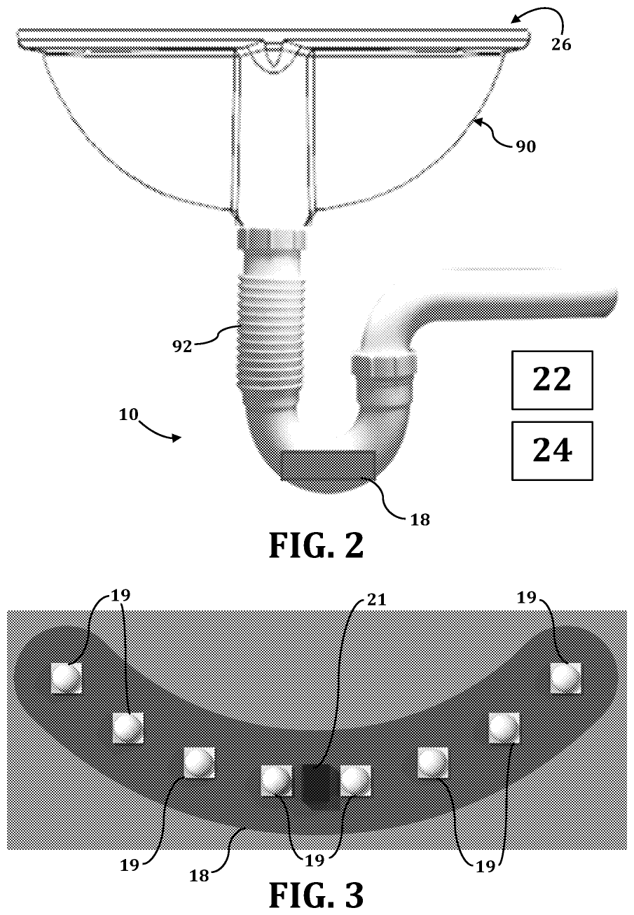

In some examples, drain disinfection system 10 can operate with sink systems having one or more disinfection indicators 26 positioned proximate the drain within basin 90 or near the faucet 95 (e.g., as depicted in FIG. 2). Disinfection indicators 26 can indicate when system 10 is turned on or turned off. For example, disinfection indicators 26 can provide visual, tactile, or audible feedback to a user by activating in conjunction with system 10. Conversely, disinfection indicators 26 can be deactivated in conjunction with system 10 to indicate to a user that system 10 is turned off. This advantageously allows users to know the immediate status of system 10 without having to directly observe operation below the sink deck or another location where disinfection module 18 is located (or other components that can provide an indication of use, e.g., power unit 14). In some examples, indicators 26 can be flashing lights, an audible noise, or another discernable indicator of the operational status of system 10.

Accordingly, drain disinfection system 10 can eliminate potentially infectious microorganisms that grow in drains. This prevents the growth of biofilms and the potential subsequent release of potentially infectious aerosols into the surrounding environment. System 10 effectively disinfects both drain surfaces and fluids using ultraviolet light, including eliminating pathogens such as E. coli, Salmonella, and Staphylococcus aureus. This not only reduces the risk of infections and illnesses caused by aerosolized microorganisms, but also ensures a cleaner and safer sink environment. The ability to operate near or within the “p-trap” section of the drain minimizes human exposure to ultraviolet light. The programmable functionality allows for tailored disinfection schedules and disinfection intensity adjustments based on contamination levels. Additionally, features like sensors, display units, and indicators in the basin 90 or proximate the faucet 95 enhance user convenience and provide real-time feedback on system performance. These features make the drain disinfection system 10 advantageous for maintaining hygiene and reducing health risks in sink systems.

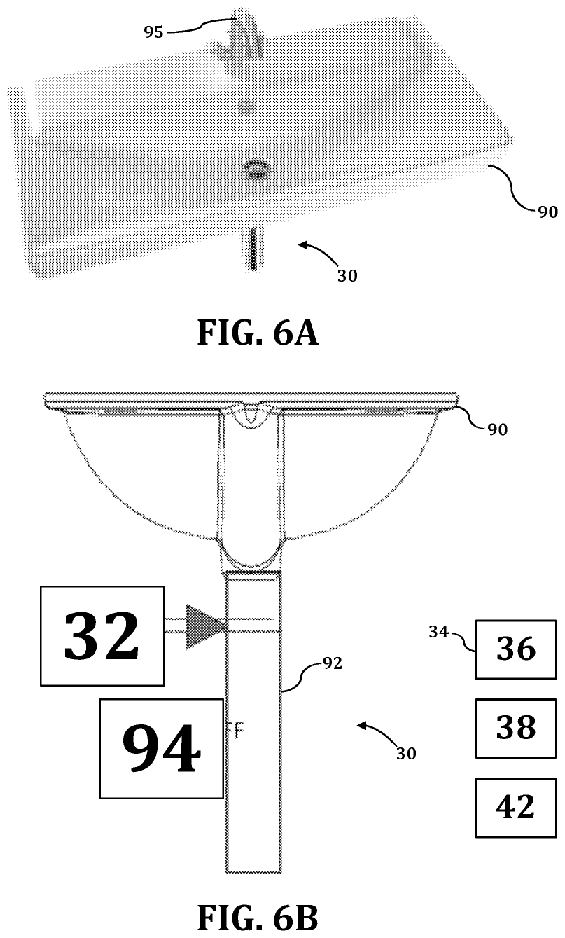

FIGS. 6A and 6B depict a drain disinfection system 30 used with basin 90 and faucet 95, according to examples of the present disclosure. Similar to system 10, drain disinfection system 30 can eliminate potentially infectious microorganisms located in a drain or drain passageway. System 30 can achieve this using low intensity electric pulses that can stimulate surfaces where microorganisms are growing, causing them to dislodge and be removed from the drain passageway. Because the electric pulses are low intensity, they are not harmful to humans and thus are suitable for use with sink systems in residential settings. In some examples, system 30 can be used with sink systems having a one-way or two-way valve 94 (similar to system 10).

For simplicity, certain descriptions of common components are not repeated in the following in as complete detail as discussed previously. Like reference numerals and feature names may designate like feature names throughout that are corresponding or analogous. For example, as with system 10, system 30 can include a power unit stored within a power unit housing, a disinfection module in the form of an electric cell, a controller couplable to the electric cell, and a display for monitoring performance of the electric cell or the power unit. Such common components can be configured to operate similarly between systems 10 and 30, in line with the forthcoming description.

Drain disinfection system 30 can include an electric cell 32 which is optionally located in an electric cell housing (not depicted). The electric cell 32 is configured to output low intensity electric pulses to dislodge microorganisms from the drain. Electric cell 32 can be programmed to turn on or turn off at regularly scheduled intervals. In some examples, the intervals can be programmed by a user of system 30. In some examples, electric cell 32 can be programmed or controlled using a controller 38. Controller 38 is substantially identical to controller 22 used with system 10 and can be positioned in a similar location on the drain system (e.g., proximate the electric cell 32 or external to the drain or sink system).

System 30 can further include a power unit 36 received within a power unit housing 34. Power unit housing 34 can be coupled to or integrally formed with drain piping or tubing 92 of a sink system (e.g., sink system including system 30, basin 90, and faucet 95, among other components). Power unit housing 34 and the optional electric cell housing are generally waterproof. This protects these components from fluids flowing through the drain. In some examples, power unit 36 can be a battery or battery pack that is rechargeable (e.g., wired or wireless charging) or replaceable with a new battery or battery pack. In other examples, power unit 36 can be an electrical motor or a mechanical motor, hydraulically or pneumatically powered, powered by a renewable energy source, or capable of outputting power received from an external power source suited for use with sink systems. Power unit 36 can be configured to continuously output power to electric cell 32 until turned off. In some examples, power unit 36 can include a device to store energy for future use with system 30.

System 30 can further include a display unit 42 to indicate the functioning of electric cell 32, power unit 36, and controller 38. For example, display unit 42 can output various disinfection characteristics such as whether the electric cell 32 is currently operating, when it last operated, statistics on microorganism elimination, system operating mode, ultraviolet LED or other lighting component arrangement or pattern, operational errors with electric cell 32, and other similar characteristics. Display unit 42 can be positioned external to the sink system, for example near faucet 95, or can be positioned below a sink deck proximate drain piping or tubing 92.

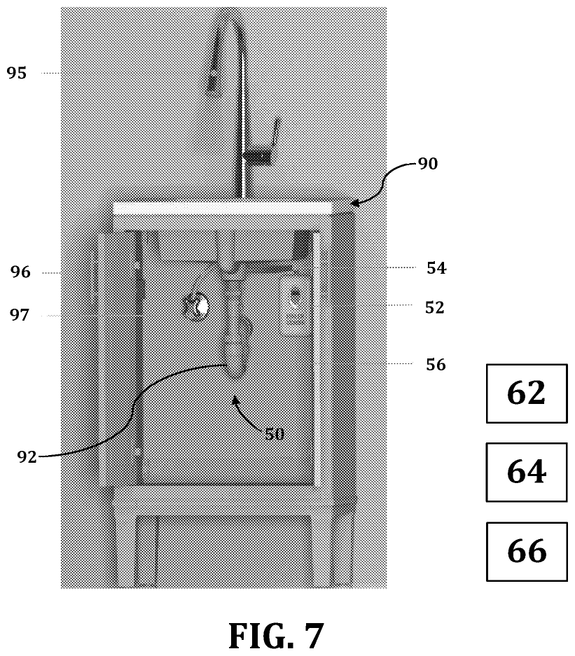

FIG. 7 depicts a drain disinfection system 50 used with basin 90 and faucet 95, according to examples of the present disclosure. Similar to systems 10 and 30, drain disinfection system 50 can eliminate potentially infectious microorganisms located in a drain or drain passageway. System 50 can achieve this by using an ozone generator to release ozone into fluid flowing through the drain. In some examples, a diverter valve is used to direct ozone from the ozone generator to the faucet 95 or to the drain piping depending on whether drain disinfection is required. The ozone generated by the ozone generator can eliminate any microorganisms within the drain by reacting with them via the ozonated fluid within the drain passageway (e.g., in piping or tubing 92 including the “p-trap” section). Like systems 10 and 30, this prevents the release of microorganism aerosolization into the environment outside the drain. Human health and safety is improved as a result.

For simplicity, certain descriptions of common components are generally not repeated in the following in as complete detail as discussed previously. Like reference numerals and feature names may designate like feature names throughout that are corresponding or analogous. For example, as with systems 10 and 30, system 50 can include a power unit 62 stored within a power unit housing, a disinfection module in the form of an ozone generator 52, a controller 64 couplable to the ozone generator 52, and a display 66 for monitoring performance of the ozone generator 52 or the power unit 62. In some examples, system 50 does not include a separate power unit or any additional wiring because system 50 is connected to an external power source included with the sink system. Such common components can be configured to operate similarly between systems 10, 30, and 50, in line with the forthcoming description.

Drain disinfection system 50 can include an ozone generator 52 configured to generate and release ozone into fluid flowing through a drain. Ozone can be released through an ozone diffusion hose 54 couplable to the drain. Ozone diffusion hose 54 can couple ozone generator 52 (which is generally positioned below a sink deck) to a drain or drain passageway of a sink system (e.g., piping or tubing 92 including the “p-trap” section). Generally, a diverter valve is positioned within the drain or drain passageway. The diverter valve can direct fluid to the faucet 95 or to the ozone generator 52 if drain disinfection is needed. The diverter valve can be coupled to a fluid inlet valve 97 via a faucet inlet tube 96, and can be coupled to the ozone generator 52 via ozone diffusion hose 54. As such, fluid can be directed to the faucet 95 or to the ozone generator 52 through ozone diffusion hose 54.

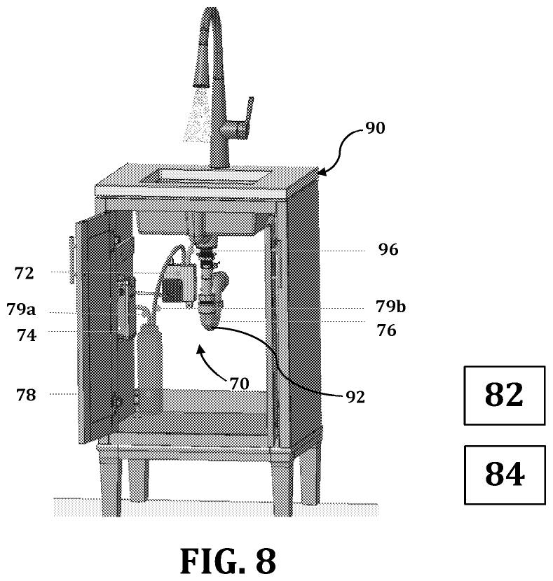

When disinfection is required, fluid can first optionally be diverted to a sediment filter that can remove debris and other impurities in the fluid (illustrated in FIG. 8 and described in further detail below). After being cleansed in the sediment filter, fluid can flow to the ozone generator 52 where ozone is produced and released into the fluid. The ozonated fluid can then be released into the drain where microorganisms may be located. The ozonated fluid reacts with the microorganisms on the drain surfaces to eliminate them, thereby preventing aerosolization outside of the drain. In some examples, the ozone generator 52 can be programmed to be turned on or turned off at particular times of the day or when certain circumstances exist (e.g., whenever fluid is flowing through the drain passageway). Programming can be achieved using controller 64 coupled (e.g., operably, electrically, communicatively) to the ozone generator 52 in some examples, or ozone generator 52 can be programmed directly and operated without a separate controller in other examples. Display unit 66 can be used to select the disinfection mode or disinfection characteristics used by ozone generator 52 (in conjunction with other components of system 50).

In some examples, sink systems with basin 90, faucet 95, and system 50 can operate with a fluid flow rate of about 5.7 liters per minute (about 1.5 gallons per minute) at a pressure of about 4.14 bar (about 60 pounds per square inch). In some examples, system 50 does not include a separate power unit or any additional wiring because system 50 uses an external power source to power the components (e.g., ozone generator 52, controller 64, display unit 66). Such examples provide a more compact configuration for system 50 because less space is used below the sink deck or wherever system 50 is located relative to basin 90 and faucet 95.

FIG. 8 depicts a drain disinfection system 70 used with basin 90 and faucet 95, according to examples of the present disclosure. Similar to systems 10, 30, and 50, drain disinfection system 70 can eliminate potentially infectious microorganisms located in a drain or drain passageway. System 70 can achieve this by using an electrolyzation cell to electrolyze fluid flowing through the drain. In some examples, a diverter valve is used to direct fluid to the faucet 95 or to the electrolyzation cell depending on whether drain disinfection is required. Electrolyzation can eliminate any microorganisms within the drain by reacting with them via the electrolyzed fluid flowing through the drain passageway (e.g., in piping or tubing 92 including the “p-trap” section 76). Like systems 10, 30, and 50, this prevents the release of aerosolized microorganism into the environment outside the drain.

For simplicity, certain descriptions of common components are generally not repeated in the following in as complete detail as discussed previously. Like reference numerals and feature names may designate like feature names throughout that are corresponding or analogous. For example, as with systems 10, 30, and 50, system 70 can include a power unit stored within a power unit housing, a disinfection module in the form of an electrolyzation cell, a controller couplable to the electrolyzation cell, and a display for monitoring performance of the electrolyzation cell or the power unit. Such common components can be configured to operate similarly between systems 10, 30, and 50, in line with the forthcoming description.

Drain disinfection system 70 can include an electrolyzation cell 72 configured to electrolyze fluid flowing through a drain. Electrolyzation cell 72 can be coupled to a faucet inlet tube 96 for transporting electrolyzed fluid (i.e., electrolytic water or “e-water”) to the faucet 95. Generally, a diverter valve is positioned within the drain or drain passageway. The diverter valve can direct fluid to the faucet 95 or to the electrolyzation cell 72 if drain disinfection is needed. The diverter valve can be coupled to a fluid inlet valve (not depicted) via the faucet inlet tube 96, and can be coupled to the electrolyzation cell 72 also via the faucet inlet tube 96 (or other tubing or piping).

In some examples, system 70 can include an optional sediment filter 78 to remove debris and impurities from fluid before the fluid is electrolyzed by the electrolyzation cell 72. Sediment filter 78 can include an optional inlet tube 79a couplable to electronic unit 74 and an optional outlet tube 79b couplable to electrolyzation cell 72. After debris and impurities are removed from fluid at the optional sediment filter 78, the cleansed fluid can travel to the electrolyzation cell 72 via the optional outlet tube 79b to be electrolyzed. Microorganisms in the drain or drain passageway can be eliminated by the electrolyzed fluid traveling therein, thus preventing the release of harmful aerosolization to the outside environment. It should be understood that optional sediment filter 78 can be included with systems 10, 30, 50 in a similar manner as with system 70 (e.g., fluid can be cleansed in system 50 prior to being ozonated by the ozone generator 52).

After being cleansed with the optional sediment filter 78, fluid can flow to the electrolyzation unit 72 where the fluid is electrolyzed. The electrolyzed fluid can then be released into the drain where microorganisms may be located. The electrolyzed fluid reacts with any microorganisms on the drain surfaces to eliminate them, thereby preventing aerosolization outside of the drain. In some examples, the electrolyzation unit 72 can be programmed to be turned on or turned off at particular times of the day or when certain circumstances exist (e.g., whenever fluid is flowing through the drain passageway). Programming can be achieved using controller 82 coupled (e.g., operably, electrically, communicatively) to the electrolyzation unit 72 in some examples, or electrolyzation unit 72 can be programmed directly and operated without a separate controller in other examples. Display unit 84 can be used to select or view the disinfection mode or disinfection characteristics used by the electrolyzation unit 72 (in conjunction with other components of system 70).

In some examples, sink systems with basin 90, faucet 95, and system 70 can operate with a fluid flow rate of about 5.7 liters per minute (about 1.5 gallons per minute) at a pressure of about 4.14 bar (about 60 pounds per square inch). In some examples, system 70 does not include a separate power unit or any additional wiring because system 70 uses an external power source to power the components (e.g., electrolyzation unit 72, controller 82, display unit 84). Such examples provide a more compact configuration for system 70 because less space is used below the sink deck or wherever system 70 is located relative to basin 90 and faucet 95.

Accordingly, examples of the present disclosure provide drain systems having sanitization or disinfection functionality to eliminate potentially infectious microorganisms. Some examples can include a disinfection module operating as a PCB with surface-mounted ultraviolet LEDs or other lighting components, an electric cell for eliminating microorganisms using low intensity electric pulses, an ozone generator for generating ozonated fluid, and an electrolyzation cell for generating electrolyzed fluid. Ozonated and electrolyzed fluid can be used to eliminate microorganisms growing in drain systems.

In some examples of any of the systems or techniques described herein, the following disinfection technologies, techniques, or compositions can be used to achieve drain disinfection (via a disinfection module, an electric cell, an ozone generator, an electrolyzation cell, a sediment filter, or another module or unit configured to disinfect, sanitize, or filter drains and drain passageways): ultraviolet LEDs arranged along a PCB strip positioned in the “p-trap” of drain piping or tubing, or another section; nanobubbles; ozone disinfection; electrolyzed fluid via the electrolyzation cell; ultra-sonification; thermal disinfection; pulsed electric field (low intensity); ultraviolet germicidal irradiation (UVGI); hydrogen peroxide; chlorine dosing; a combination of ozone and UVGI disinfection; sodium hypochlorite; high temperature steam disinfection; microwave disinfection; plasma incineration; air, carbon dioxide, or other gas-burst drain cleaners to disintegrate or dislodge microorganisms (or to clear a drain clog); hydro-jetting drain cleaners; electric or bioelectric disinfection; non-thermal plasma; or combinations thereof.

Some of these disinfection technologies, techniques, and compositions were described previously in greater detail with systems 10, 30, 50, 70. However, it should be understood that any of these disinfection technologies, techniques, and compositions can be used with systems 10, 30, 50, 70 to enable disinfection as described herein. Combinations of such technologies, techniques, and compositions are also contemplated by the present disclosure.

The disclosure may be embodied in other specific forms without departing from the essential attributes. Therefore, the illustrated examples should be considered illustrative and not restrictive in all respects. Any claims provided herein are to ensure adequacy of the present application for establishing foreign priority and for no other purpose.

Various examples of systems, devices, and methods have been described herein. These examples are given only be way of example and are not intended to limit the scope of the claimed disclosures. It should be appreciated, moreover, that the various features of the examples that have been described may be combined in various ways to produce numerous additional examples. Moreover, while various material, dimensions, shapes, configurations, locations, etc. have been described for use with disclosed examples, others besides those disclosed may be utilized without exceeding the scope of the claimed disclosures.

Persons of ordinary skill in the relevant arts will recognize that the subject matter hereof may comprise fewer features than illustrated in any individual example described above. The examples described herein are not meant to be an exhaustive presentation of the ways in which the various features of the subject matter hereof may be combined. Accordingly, the examples are not mutually exclusive combinations of features; rather, the various examples can comprise a combination of different individual features selected from different individual examples, as understood be persons of ordinary skill in the art. Moreover, elements described with respect to one example can be implemented in other examples even when not described in such examples unless otherwise noted.

Any incorporation of reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A drain disinfection system, comprising:
a disinfection module including at least one ultraviolet light-emitting component configured to emit ultraviolet light to sanitize drain piping by eliminating microorganisms inside the drain piping;
a power unit configured to supply power to the disinfection module; and
a controller configured to control operation of the disinfection module,
wherein the disinfection module is positioned proximate a p-trap section of the drain piping to eliminate microorganisms growing in the p-trap section and present in fluids flowing through the drain piping.

2. The drain disinfection system of claim 1, wherein the power unit is contained within a first housing coupled to the drain piping.

3. The drain disinfection system of claim 1, wherein the disinfection module is contained within a second housing coupled to the drain piping.

4. The drain disinfection system of claim 1, wherein the disinfection module comprises a printed circuit board including a plurality of ultraviolet light-emitting diodes.

5. The drain disinfection system of claim 4, wherein at least one of the ultraviolet light-emitting diodes is configured to operate in a wavelength range of 180 to 222 nanometers.

6. The drain disinfection system of claim 5, wherein at least one of the ultraviolet light-emitting diodes is configured to operate in a wavelength range of 260 to 280 nanometers.

7. The drain disinfection system of claim 6, wherein the plurality of ultraviolet light-emitting diodes are arranged in an alternating pattern of wavelengths to vary the intensity of ultraviolet light emitted from the disinfection module.

8. The drain disinfection system of claim 1, wherein the controller is coupled to the disinfection module and configured to activate or deactivate the disinfection module.

9. The drain disinfection system of claim 8, further comprising a sensor configured to sense when fluid flows through the drain piping, wherein the sensor is coupled to the controller.

10. The drain disinfection system of claim 9, wherein the controller is configured to activate the disinfection module in response to an indication from the sensor that fluid is flowing through the drain piping.

11. The drain disinfection system of claim 1, further comprising a disinfection indicator positioned proximate a basin associated with the drain disinfection system, the disinfection indicator configured to indicate an operation status of the drain disinfection system.

12. A drain disinfection system, comprising:
a disinfection module including ozone generator component configured to emit ozone to sanitize drain piping by eliminating microorganisms inside the drain piping;
a power unit configured to supply power to the disinfection module; and
a controller configured to control operation of the disinfection module,
wherein the disinfection module is coupled to the drain piping via a diffusion hose.

13. The drain disinfection system of claim 12, further comprising a diverter valve configured to direct ozone to the drain piping or to a faucet associated with the drain disinfection system.

14. The drain disinfection system of claim 12, wherein the controller is coupled to the disinfection module and configured to activate or deactivate the disinfection module.

15. The drain disinfection system of claim 14, further comprising a sensor configured to sense when fluid flows through the drain piping, wherein the sensor is coupled to the controller.

16. The drain disinfection system of claim 15, wherein the controller is configured to activate the disinfection module in response to an indication from the sensor that fluid is flowing through the drain piping.

17. The drain disinfection system of claim 12, wherein the ozone generator is positioned proximate the drain piping.

18. The drain disinfection system of claim 12, further comprising a display unit configured to indicate one or more functional characteristics of the disinfection module, power unit, or controller.

19. The drain disinfection system of claim 12, further comprising a disinfection indicator positioned proximate a basin associated with the drain disinfection system, the disinfection indicator configured to indicate an operation status of the drain disinfection system.

20. A drain disinfection system, comprising:
a disinfection module including at least one ultraviolet light-emitting component configured to emit ultraviolet light to sanitize drain piping by eliminating microorganisms inside the drain piping, wherein the disinfection module is contained within a first housing coupled to the drain piping, and wherein the disinfection module comprises a printed circuit board including a plurality of ultraviolet light-emitting diodes, wherein at least one of the ultraviolet light-emitting diodes is configured to operate in a wavelength range of 180 to 280 nanometers;
a power unit configured to supply power to the disinfection module, wherein the power unit is contained within a second housing coupled to the drain piping;
a controller configured to control operation of the disinfection module, wherein the controller is coupled to the disinfection module and configured to activate or deactivate the disinfection module;
a sensor configured to sense when fluid flows through the drain piping, wherein the sensor is coupled to the controller, and wherein the controller is configured to activate the disinfection module in response to an indication from the sensor that fluid is flowing through the drain piping
wherein the disinfection module is positioned proximate a p-trap section of the drain piping to eliminate microorganisms growing in the p-trap section and present in fluids flowing through the drain piping.
